(19) United States
(12) Patent Application Publication
(10) Pub. No.: US 2025/0163100 A1
(43) Pub. Date: May 22, 2025

(54) METHOD FOR RECOVERING OF HIGH PURITY BETULIN FROM BIRCH BARK

(71) Applicant: Innomost Oy, Kokkola (FI)

(72) Inventor: Sami Selkälä, Kokkola (FI)

(73) Assignee: Innomost Oy, Kokkola (FI)

(21) Appl. No.: 18/839,664

(22) PCT Filed: Feb. 23, 2023
(86) PCT No.: PCT/FI2023/050105

§ 371 Date: Aug. 19, 2024

(30) Foreign Application Priority Data
Feb. 24, 2022 (FI) 20225170

(51) Int. Cl. C07J 63/00

(52) U.S. Cl. CPC C07J 63/008

(57) ABSTRACT

A method for recovering high purity betulin from birch bark is disclosed.

### TECHNICAL FIELD

The present disclosure relates to a method for recovering high purity betulin from birch bark.

### BACKGROUND

Birch (Betula spp.) is widespread throughout the Northern Hemisphere and harvested in huge volumes. Birch bark is produced in considerable quantities as a byproduct of the forest industry and its upgrading is almost totally neglected. Currently, this low-value side stream is burnt for combined heat and power production.

Birch bark could, however, find more valuable uses, for example as an additive in plastic composite materials. Birch bark consists of brown inner bark ˜75% and white outer bark ˜25%. The inner bark consists mainly of wood-like material such as lignin, pentosans and hexosans. The outer bark contains, by dry weight, up to 40% fats, fatty acids, resins and triterpenes, in particular betulin, at up to 30%. In addition, the outer bark contains up to 35% suberin. Valorization and upgrading of these compounds by using modern chemical technology opens up entirely new opportunities to produce new specialty chemicals from this low-value biomass stream.

It has been estimated that a pulp mill with an annual production capacity of 200,000 tonnes of birch Kraft pulp produces enough bark to produce around 2,500 tonnes of betulin of around 95% purity per year. New potential applications for betulin or betulin derivatives include pharmaceuticals and cosmetic products as well as agrochemicals.

### SUMMARY

A method for recovering high purity betulin from birch bark is disclosed. The method may comprise:
providing a raw material comprising birch bark,
screening the raw material comprising birch bark to remove fines to form a screened raw material,
separating the raw material into a fraction comprising inner bark and wood particles, and a fraction comprising outer bark,
drying the fraction comprising outer bark to form dried bark,
cutting the dried bark in a cutting mill to form cut bark,
screening the cut bark to remove fines and form a fraction comprising bark,
milling the fraction comprising bark in a cutting milling to form a bark powder,
extracting betulin from the bark powder, and
purifying the betulin to form high purity betulin.

### BRIEF DESCRIPTION OF THE DRAWINGS

The accompanying drawing, which is included to provide a further understanding of the embodiments and constitute a part of this specification, illustrates various embodiments. In the drawings:

FIG. 1 presents a flow chart of one embodiment of the method for recovering of high purity betulin from birch bark.

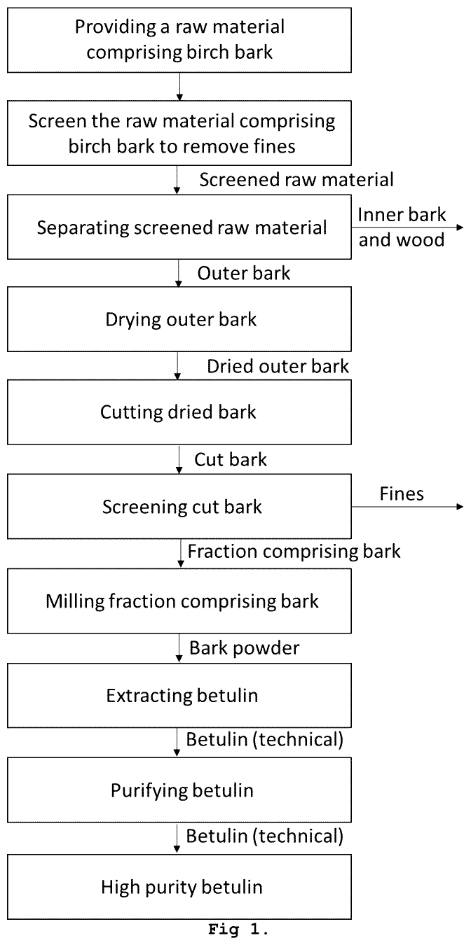

### DETAILED DESCRIPTION

Birch bark can broadly be said to consist of two distinct layers: an inner dark layer and an outer light layer. While the inner layer is wood-like and generally burned for energy. The outer, light or white, layer has been found to contain several different extractives that have different potential uses if extracted from the bark.

A method for recovering high quality betulin from birch bark is disclosed. The method may comprise:
providing a raw material comprising birch bark,
screening the raw material comprising birch bark to remove fines to form a screened raw material,
separating the screened raw material into a fraction comprising inner bark and wood particles, and a fraction comprising outer bark,
drying the fraction comprising outer bark to form dried bark,
cutting the dried bark in a cutting mill to form cut bark,
screening the cut bark to remove fines and form a fraction comprising bark,
milling the fraction comprising bark in a cutting milling to form a bark powder,
extracting betulin from the bark powder, and
purifying the betulin to form high purity betulin.

Birch bark may be acquired e.g. as side products or wastes from other processes of forest or paper industry. E.g. wood intended for the production of paper is commonly debarked to form debarked logs and a fraction containing a mixture of bark particles and wood removed from the logs in the debarking. If the raw logs are to be sawed to be used as e.g. construction material, the outermost parts including the bark layers is commonly discarded and may in some cases be burned for energy.

When the logs are debarked, the bark is separated from the logs to form a mixture of particles of various size that include both outer bark, inner bark, and some wood that has been separated from the logs. In the mixture of particles some of the outer bark, inner bark, and wood will be separate particles and some will be separated into the various components.

In one embodiment, the betulin is extracted from the bark powder using liquid-solid extraction.

In one embodiment, screening the raw material comprising birch bark comprises screening the raw material comprising birch bark in a drum screening to remove particles with a particle size of 45 mm or less. In certain embodiments, screening the raw material comprising birch bark comprises screening the raw material comprising birch bark in a drum screening to remove particles with a particle size of 45 mm or less, or 40 mm or less, or 35 mm or less, or 30 mm or less, or 25 mm or less.

In one embodiment, separating the screened raw material into a fraction comprising inner bark and wood particles, and a fraction comprising outer bark comprises sorting the screened raw material optically to separate inner bark and wood particles from outer bark.

The inner bark after sorting also comprises moisture from the original raw material. In order to improve the efficiency of the extraction, the bark material fed into the extraction should contain as little moisture as possible.

In one embodiment, drying the fraction comprising outer bark comprises subjecting it to at least one drying using warm air.

The efficiency of the extraction is also dependent on the particle size of the solid material on which the extraction is performed. When the particle size is reduced, the relative surface area is increased which improves the efficiency of the extraction.

In certain embodiments, the dried bark is milled in a cutting mill to a particle size of 6 mm-45 mm. In one embodiment, the dried bark is milled in a cutting mill to a particle size of approximately 6 mm-45 mm, or approximately 6 mm-40 mm, or approximately 6 mm-35 mm, or approximately 6 mm-25 mm, or approximately 6 mm-20 mm. In one embodiment, the dried bark is milled in a cutting mill to a particle size of approximately 45 mm or less.

By milling the dried bark in a cutting mill, it is possible to cut, mill, or grind the inner bark still attached to the outer bark into small particles that are separated from the outer bark. Once dried, the inner bark becomes brittle and is thus ground into fine particles in the milling. As the brittle inner bark is ground into finer particles than the outer bark, it is possible to separate the inner bark particles from the outer bark by screening the cut bark.

As will be obvious to a person skilled in the art, cutting or milling dried bark to a given size will produce a mixture of particles of various sizes. While the largest particles present in the resulting mixture will have the desired size, the mixture will inevitably also comprise particles of smaller size. As a non-limiting example, milling dried bark in a cutting mill to a particle size of approximately 45 mm may produce a mixture of particles in a size range of approximately 6 mm-45 mm.

In one embodiment, the cut bark is screened to remove particles of a size of approximately 6 mm or smaller. In certain embodiments, the cut bark is screened to remove particles of a size of approximately 6 mm or smaller, or approximately 5 mm or smaller, or approximately 4 mm or smaller, or approximately 3 mm or smaller.

In one embodiment, the fraction comprising bark is milled to a particle size of 0 mm-6 mm. In certain embodiments, the fraction comprising bark is milled to a particle size of 0 mm-6 mm, or 0 mm-5 mm, or 0 mm-4 mm, or 0 mm-3 mm, or 0 mm-2.5 mm, or 0 mm-2 mm, or 0 mm-1.5 mm, or 0 mm-1.0 mm, or 0 mm-0.5 mm.

Once the bark material has been processed into particle with a suitable particle size, the bark powder may be subjected to extractions and other chemical treatments to obtain chemical compounds and products for further use.

In certain embodiments, various chemical compounds may be extracted from birch bark powder directly using liquid-solid extraction using a suitable solvent. The solvent and the extraction process may be selected according to the compound to be extracted.

In one embodiment, the method further comprises subjecting the bark powder to a liquid-solid extraction to recover betulin from the bark powder.

In one embodiment, the method for extracting betulin from the bark powder comprises extracting the bark powder with an organic solvent under reflux conditions followed by isolation of the betulin by consecutive decantation and reslurrying steps. After removal of excess solvent, betulin may be collected.

In certain embodiments, the method for extracting betulin from the bark powder comprises extracting the bark powder with a solvent selected from methanol, ethanol, 1-propanol, isopropanol, 1-butanol, 2-butanol, 1-pentanol, 2-pentanol, 1-hexanol, methyl acetate, ethyl acetate, propyl acetate, isopropyl acetate, butyl acetate, cyclohexane, methyl cyclohexane, toluene, tetrahydrofuran, 2-methyltetrahydrofuran, methyl ethyl ketone, acetone, 2,2,5,5-tetramethyloxolane, 2-methoxyethanol, or any combination thereof under reflux conditions followed by isolation of the betulin by consecutive decantation and reslurrying steps. After removal of excess solvent, the betulin may be collected.

In one embodiment, the betulin extraction is performed using a solvent selected from methanol, ethanol, 1-propanol, isopropanol, 1-butanol, 2-butanol, 1-pentanol, 2-pentanol, 1-hexanol, methyl acetate, ethyl acetate, propyl acetate, isopropyl acetate, butyl acetate, cyclohexane, methyl cyclohexane, toluene, tetrahydrofuran, 2-methyltetrahydrofuran, methyl ethyl ketone, acetone, 2,2,5,5-tetramethyloxolane, 2-methoxyethanol, or any combination thereof heated to reflux followed by decantation of the solution comprising dissolved betulin. After removal of excess solvent, the betulin may be collected.

In one embodiment, the method for extracting betulin from the bark powder comprises extracting the bark powder with isopropanol under reflux conditions followed by isolation of the betulin by consecutive decantation and reslurrying steps. In one embodiment, the betulin extraction is performed using isopropanol heated to reflux followed by decantation of the isopropanol solution comprising dissolved betulin. After removal of excess solvent, the betulin may be collected.

In certain embodiments, the purity of the betulin collected after the extraction may be at least 80%, or at least 83%, or at least 85%, or at least 88%, or at least 90%, or at least 93%.

In one embodiment, the liquid-solid extraction to recover betulin comprises extraction with isopropanol followed by isolation of the betulin.

In one embodiment, the method comprises extracting betulin performed using a first extraction performed using an organic solvent heated to reflux followed by decantation of the solution comprising dissolved betulin followed by a second extraction of the solids formed in the first extraction using the same solvent heated to reflux followed by decantation of the solution comprising betulin which is combined with the solution comprising betulin formed in the first extraction and the combined solution comprising betulin is filtered to remove small particles.

In one embodiment, the method comprises extracting betulin performed using a first extraction performed using an organic solvent selected from methanol, ethanol, 1-propanol, isopropanol, 1-butanol, 2-butanol, 1-pentanol, 2-pentanol, 1-hexanol, methyl acetate, ethyl acetate, propyl acetate, isopropyl acetate, butyl acetate, cyclohexane, methyl cyclohexane, toluene, tetrahydrofuran, 2-methyltetrahydrofuran, methyl ethyl ketone, acetone, 2,2,5,5-tetramethyloxolane, 2-methoxyethanol, or any combination thereof heated to reflux followed by decantation of the solution comprising dissolved betulin followed by a second extraction of the solids formed in the first extraction using the same solvent heated to reflux followed by decantation of the solution comprising betulin which is combined with the solution comprising betulin formed in the first extraction and the combined solution comprising betulin is filtered to remove small particles.

In one embodiment, the method comprises extracting betulin performed using a first extraction performed using isopropanol heated to reflux followed by decantation of the isopropanol solution comprising dissolved betulin followed by a second extraction of the solids formed in the first extraction using isopropanol heated to reflux followed by decantation of the isopropanol solution comprising betulin which is combined with the isopropanol solution comprising betulin formed in the first extraction and the combined isopropanol solution comprising betulin is filtered to remove small particles.

In one embodiment, the method comprises recovering betulin from the solution comprising betulin by distilling off excess solvent followed by cooling the solution to room temperature to allow the betulin to crystallize, decanting excess solvent, reslurrying the betulin by the addition of fresh solvent and allowing the slurry to settle, decanting excess solvent to recover betulin, and drying the betulin crystals.

In one embodiment, the method comprises recovering betulin from the solution comprising betulin by distilling off excess solvent at atmospheric pressure followed by cooling the solution to room temperature to allow the betulin to crystallize, decanting excess solvent, reslurrying the betulin by the addition of fresh solvent and allowing the slurry to settle, decanting excess solvent to recover betulin, and drying the betulin crystals.

In one embodiment, the method comprises recovering betulin from the isopropanol solution comprising betulin by distilling off excess isopropanol at atmospheric pressure followed by cooling the isopropanol to room temperature to allow the betulin to crystallize, decanting excess solvent, reslurrying the betulin by the addition of isopropanol and allowing the slurry to settle, decanting excess solvent to recover betulin, and drying the betulin crystals.

The betulin crystals may be dried at any temperature and pressure suitable. A person skilled in the art will be able to determine suitable drying conditions e.g. based on the solvent used. In certain embodiments, the betulin crystals may be dried at room temperature or elevated temperature, and at atmospheric pressure or reduced pressure. In certain embodiments, the betulin crystals may be dried at a temperature of approximately 50-120° C. and a pressure of 20-500 mbar. In certain embodiments, the betulin crystals may be dried by lyophilization or freeze drying.

In certain embodiments, the method comprises purifying the betulin by suspending the betulin in aqueous base solution and heating the suspension to reflux, cooling the suspension to below its boiling point, adding a suitable organic solvent that forms azeotropes with water to the suspension, removing water and solvent by azeotropic distillation, filtering the solution of betulin while hot, distilling off part of the solvent at atmospheric pressure, cooling the suspension to room temperature to allow the betulin to crystallize, filtering the solution to recover the betulin crystals, washing the recovered betulin crystals with excess solvent, and drying the betulin crystals to recover betulin at a purity of 98% or more.

In certain embodiments, the method comprises purifying the betulin by suspending the betulin in aqueous base solution and heating the suspension to reflux, cooling the suspension to below its boiling point, adding a suitable organic solvent that forms azeotropes with water selected from cyclohexane, methyl cyclohexane, tetrahydrofuran, 2-methyltetrahydrofuran, 2,2,5,5-tetramethyloxolane, toluene, or any combination thereof to the suspension, removing water and solvent by azeotropic distillation, filtering the solution of betulin while hot, distilling off part of the solvent at atmospheric pressure, cooling the suspension to room temperature to allow the betulin to crystallize, filtering the solution to recover the betulin crystals, washing the recovered betulin crystals with excess solvent, and drying the betulin crystals to recover betulin at a purity of 98% or more.

In one embodiment, the method comprises purifying the betulin by suspending the betulin in aqueous base solution and heating the suspension to reflux, cooling the suspension to below its boiling point, adding toluene to the suspension, removing water and toluene by azeotropic distillation, filtering the solution of betulin in toluene while hot, distilling off part of the toluene at atmospheric pressure, cooling the toluene suspension to room temperature to allow the betulin to crystallize, filtering the solution to recover the betulin crystals, washing the recovered betulin crystals with excess toluene, and drying the betulin crystals to recover betulin at a purity of 98% or more.

In one embodiment, the solvent recovered from the process is recycled for re-use in the process.

The method for recovering of high purity betulin from birch bark described in the current specification has the added utility of reducing the total amount of energy required to obtain high purity betulin starting from birch bark. Removing the inner bark and wood usually included in bark fractions, the amount of moisture is included in the bark is reduced significantly, meaning that less energy is required for drying the bark. Additionally, producing high quality technical betulin using the method of the present application simplifies the purification the betulin by forming a technical product with a favourable impurity profile.

An additional advantage is that as the outer bark is removed from the bark fraction, the amount of material fed into the liquid-solid extraction may be reduced significantly, providing savings in energy and reducing the amount of solvent required for the extraction itself. A still further advantage of the methods disclosed herein is that all solvents used can be recycled and reused in the process, thereby lower the total amounts of harmful chemicals needed.

### EXAMPLES

Reference will now be made in detail to various embodiments.

The description below discloses some embodiments in such a detail that a person skilled in the art is able to utilize the embodiments based on the disclosure. Not all steps or features of the embodiments are discussed in detail, as many of the steps or features will be obvious for the person skilled in the art based on this specification.

### Example 1—Processing Bark

Approximately 550 kg (2.2 m³) of birch bark material was acquired from a veneer factory. The crude birch bark mixture included about 54 weight-% or 297 kg of dry matter. The crude birch bark material was screened using a drum screening to remove particles with a size of 25 mm or less. Approximately 176 kg of material with a particle size of 25 mm or less was collected and discarded. Approximately 374 kg of wet material with a particle size of over 25 mm was collected. The wet material collected included both wood and bark pieces, including both inner and outer bark.

The collected wet material with a particle size of 25 mm or more was sorted using an optical sorting device to separate the outer bark from the inner bark and wood particles. Approximately 208 kg of inner bark and wood particles were collected and discarded. After sorting, approximately 166 kg of wet outer bark particles with a size of 25 mm or more was collected.

The wet outer bark particles were dried using a hot air drier in four consecutive drying steps. After drying, approximately 94 kg of dry outer bark with a particle size of 25 mm or more was collected. Approximately 72 kg of water was collected from the drying process.

After drying, the outer bark was milled in a cutting mill to a particle size of around 20 mm which produced a mixture of dry bark with a particle size of 0-20 mm.

After milling, the particles of dry outer bark were screened using a drum screening device to remove small particles with a size of 3 mm or less. Approximately 29 kg of particles with a size of 3 mm or less were discarded and approximately 65 kg of particles with a size of 3-20 mm were collected.

Finally, the outer bark particles with a size of 3-20 mm were ground using a cutting grinder to produce approximately 65 kg of dry bark powder with a particle size of 0-0.5 mm.

### Example 2—Extraction of Betulin

120 kg (0.55 m³) of dry birch bark powder was suspended in 820 l (643 kg) of isopropanol, heated to 81-84° C. and refluxed for 4 h. The hot suspension was decanted, and the decantation liquid filtered to remove fine particles. After decantation, 250 l of isopropanol solution containing betulin and impurities was collected. 590 l of fresh isopropanol was added to the solution which was refluxed for 4 h followed by decanting to collect approximately 560 l of isopropanol solution of crude betulin.

To isolate betulin, the decanted liquids from several extraction steps were combined to form 1150 l (913 kg) of betulin solution. The solution was heated to distill of approximately 810 l of isopropanol and allowed to cool to room temperature (RT). Once the betulin suspension had cooled to RT, 280 l of isopropanol was decanted.

After decantation, the betulin was reslurried by the addition of 70 l of isopropanol and approximately 60 l of isopropanol decanted to produce a product cake made up of approximately 25 weight-% betulin, and 75 weight-% isopropanol.

The product cake was dried at 80° C. and reduced pressure (approximately 150 mbar) for 5 h. After drying, 13 kg of technical betulin with a purity of approximately 93% was collected. Total yield of betulin from birch bark powder was about 11% based on dry mass.

All of the isopropanol collected in the process was recycled for use in the same process.

### Example 3—Purification of Betulin

140 l (141 kg) of water was charged into a reactor and 4 kg sodium hydroxide added followed by 18 kg of technical betulin from Example 1. The mixture was heated to boiling and refluxed for 4 h. The mixture was allowed to cool to approximately 85° C. and 790 l of toluene was added.

Approximately 320 l of a mixture of water and toluene was removed by azeotropic distillation to remove essentially all water from the mixture and form a suspension of dark brown solids in toluene.

The dark brown solids were filtered off from the hot suspension and a further 460 l of toluene removed by distillation at atmospheric pressure to yield a mixture of toluene and betulin.

The mixture formed was allowed to cool to room temperature during which time the betulin crystallized. The betulin was filtered off and washed with toluene.

After washing, the betulin cake was dried at 80° C. in reduced pressure (approximately 150 mbar) which yielded 15 kg of betulin with a purity of approximately 99.1%

It is obvious to a person skilled in the art that with the advancement of technology, the basic idea may be implemented in various ways. The embodiments are thus not limited to the examples described above; instead, they may vary within the scope of the claims.

The embodiments described hereinbefore may be used in any combination with each other. Several of the embodiments may be combined together to form a further embodiment. A method, or an arrangement, disclosed herein, may comprise at least one of the embodiments described hereinbefore. It will be understood that the benefits and advantages described above may relate to one embodiment or may relate to several embodiments. The embodiments are not limited to those that solve any or all of the stated problems or those that have any or all of the stated benefits and advantages. It will further be understood that reference to ‘an’ item refers to one or more of those items. The term “comprising” is used in this specification to mean including the feature(s) or act(s) followed thereafter, without excluding the presence of one or more additional features or acts.

1. A method for recovering high quality betulin from birch bark, wherein the method comprises
providing a raw material comprising birch bark,
screening the raw material comprising birch bark to remove fines to form a screened raw material,
separating the raw material into a fraction comprising inner bark and wood particles, and a fraction comprising outer bark,
drying the fraction comprising outer bark to form dried bark,
cutting the dried bark in a cutting mill to form cut bark,
screening the cut bark to remove fines and form a fraction comprising bark,
milling the fraction comprising bark in a cutting milling to form a bark powder,
extracting betulin from the bark powder, and
purifying the betulin to form high purity betulin.

2. The method of claim 1, wherein screening the raw material comprising birch bark comprises screening the raw material comprising birch bark in a drum screening to remove particles with a particle size of 45 mm or less.

3. The method of claim 1, wherein separating the screened raw material into a fraction comprising inner bark and wood particles, and a fraction comprising outer bark comprises sorting the screened raw material optically to separate inner bark and wood particles from outer bark.

4. The method of claim 1, wherein drying the fraction comprising outer bark comprises subjecting it to at least one drying using warm air.

5. The method of claim 1, wherein the dried bark is milled in a cutting mill to a particle size of approximately 6 mm-45 mm.

6. The method of claim 1, wherein the cut bark is screened to remove particles of a size of approximately 6 mm or smaller.

7. The method of claim 1, wherein the fraction comprising bark is milled to a particle size of 0-6 mm.

8. The method of claim 1, wherein betulin is extracted from the bark powder using liquid-solid extraction.

9. The method of claim 1, wherein the betulin extraction is performed using a solvent selected from methanol, ethanol, 1-propanol, isopropanol, 1-butanol, 2-butanol, 1-pentanol, 2-pentanol, 1-hexanol, methyl acetate, ethyl acetate, propyl acetate, isopropyl acetate, butyl acetate, cyclohexane, methyl cyclohexane, toluene, tetrahydrofuran, 2-methyltetrahydrofuran, methyl ethyl ketone, acetone, 2,2,5,5-tetramethyloxolane, 2-methoxyethanol, or any combination thereof heated to reflux followed by decantation of the solution comprising dissolved betulin.

10. The method of claim 1, wherein extracting betulin performed is using a first extraction performed using a solvent selected from methanol, ethanol, 1-propanol, isopropanol, 1-butanol, 2-butanol, 1-pentanol, 2-pentanol, 1-hexanol, methyl acetate, ethyl acetate, propyl acetate, isopropyl acetate, butyl acetate, cyclohexane, methyl cyclohexane, toluene, tetrahydrofuran, 2-methyltetrahydrofuran, methyl ethyl ketone, acetone, 2,2,5,5-tetramethyloxolane, 2-methoxyethanol, or any combination thereof heated to reflux followed by decantation of the solution comprising dissolved betulin followed by a second extraction of the solids formed in the first extraction using the same solvent heated to reflux followed by decantation of the solution comprising betulin which is combined with the solution comprising betulin formed in the first extraction and the combined solution is comprising betulin filtered to remove small particles.

11. The method of claim 9, wherein the extraction solvent is isopropanol.

12. The method of claim 8, wherein betulin is recovered from the solution comprising betulin by distilling off excess solvent followed by cooling the solution to room temperature to allow the betulin to crystallize, decanting excess solvent, reslurrying the betulin by the addition of fresh solvent and allowing the slurry to settle, decanting excess solvent to recover betulin, and drying the betulin crystals to recover betulin with a purity of 90% or more.

13. The method of claim 8, wherein the betulin is purified by suspending the betulin in aqueous base solution and heating the suspension to reflux, cooling the suspension to below its boiling point, adding a solvent selected from cyclohexane, methyl cyclohexane, tetrahydrofuran, 2-methyltetrahydrofuran, 2,2,5,5-tetramethyloxolane, toluene, or any combination thereof to the suspension, removing water and solvent by azeotropic distillation, filtering the solution of betulin in toluene while hot, distilling off part of the solvent at atmospheric pressure, cooling the suspension to room temperature to allow the betulin to crystallize, filtering the solution to recover the betulin crystals, washing the recovered betulin crystals with excess solvent, and drying the betulin crystals to recover betulin at a purity of 98% or more.

14. The method of claim 13, wherein the solvent used is toluene.

15. The method of claim 8, wherein the solvent recovered from the process is recycled for re-use in the process.
